(19) United States
(12) Patent Application Publication
(10) Pub. No.: US 2016/0183567 A1
(43) Pub. Date: Jun. 30, 2016

(54) METHOD FOR PREPARING LOW ANTIGENIC FOOD AND LOW ANTIGENIC FOOD PREPARED BY SAID METHOD

(71) Applicant: KOREA FOOD RESEARCH INSTITUTE, SEONGNAM-SI (KR)

(72) Inventors: HEE DON CHOI, SEOUL (KR); YUN SOOK KIM, SEOUL (KR); HO YOUNG PARK, SEOUL (KR); SANG KEUN HA, SUWON-SI (KR); SANG-HOON LEE, BUSAN (KR); HEE-DO HONG, SEONGNAM-SI (KR); HA HYUNG KIM, SEOUL (KR); HYE SEONG HWANG, SEOUL (KR); HEAJIN PARK, SEOUL (KR); YONG KON PARK, SUWON-SI (KR); IN WOOK CHOI, SEOUL (KR)

(21) Appl. No.: 15/049,778

(22) Filed: Feb. 22, 2016

Related U.S. Application Data
(63) Continuation of application No. PCT/KR2013/007741, filed on Aug. 28, 2013.

(30) Foreign Application Priority Data
Aug. 22, 2013 (KR) 10-2013-0099975

(51) Int. Cl. A23L 1/015; C07K 14/76; A23J 3/00; C07K 14/77

(52) U.S. Cl. CPC A23L 1/015; A23J 3/00; A23L 1/29; A23L 1/32; A23L 1/36; C07K 14/77; C07K 14/76; A23V 2002/00

(57) ABSTRACT

A method for preparing a low antigenic food, the method including removing a sugar linked to a glycoprotein of an allergenic food. A low antigenic food prepared by removing a sugar linked to a glycoprotein of an allergenic food. A method for preparing a low antigenic glycoprotein, the method including removing a glucose linked to a glycoprotein selected from the group consisting of ovalbumin, ovomucoid, ovotransferrin, β-conglycinin, Ara h1, and Ara h2. A low antigenic glycoprotein prepared by removing a glucose linked to a glycoprotein selected from the group consisting of ovalbumin, ovomucoid, ovotransferrin, β-conglycinin, Ara h1, and Ara h2. A low antigenic food composition including the low antigenic glycoprotein. A low antigenic cosmetic composition including the low antigenic glycoprotein as an active ingredient.

### CROSS-REFERENCE TO RELATED APPLICATIONS

The present application is a continuation of International Patent Application No. PCT/KR2013/007741, filed on Aug. 28, 2013, which claims priority from Korean Patent Application No. 10-2013-0099975, filed on Aug. 22, 2013. The disclosures of both applications are incorporated by reference as if fully set forth herein.

### BACKGROUND

1. Field

Exemplary embodiments relate to a low antigenic food and a method for preparing a low antigenic food. More particularly, exemplary embodiments relate to a method for reducing antigenicity of an allergenic food by removing a sugar linked to a glycoprotein of the allergenic food, a method for preparing a low antigenic glycoprotein, a low antigenic food, and a low antigenic glycoprotein, and a use thereof.

2. Discussion of the Background

Allergic disease is a worldwide phenomena and its prevalence continues to increase with the improvement in the quality of our lives. In Korea, allergies have already become one of the most common chronic diseases. Food allergies caused by immune responses among the abnormal responses to food additives cause many various symptoms, including gastrointestinal symptoms such as vomiting and diarrhea, skin conditions such as hives and atopic dermatitis, and bronchial asthma and systemic anaphylactic shock. Severe responses to food allergies may result in death. In addition, there is an urgent need for the development of preventive and therapeutic measures for food allergies.

The restriction of allergy sources is common for the prevention of food allergies. Considering that most allergy sources are present in protein foods such as eggs, milk, and beans, that have high nutritious values and high intake frequency, unconditional intake restriction may cause nutritional problems. Thus, there is a need for the development of allergy reduction technologies so that foods that have allergy sources can still be consumed. Various measures for reducing allergenicity (such as protein degradation by proteases, thermal treatment at high temperatures, and combinations of alkaline treatment and thermal treatment) have been attempted. However, there are problems, such as food palatability, deteriorated functionality, and degraded food quality using these techniques. Therefore, the development of complementary and alternative technologies for the problems is needed.

Glycoproteins are biomolecules composed of complexes in which glycans are linked to particular amino acids of proteins. Glycoproteins are widely distributed in animal and plant cellular tissues. Many glycoproteins are mainly present on the outer walls of cells and are widely involved in reactions on cellular surfaces. In addition, glycoproteins play a defensive role against external stimuli as an important constituent of the cellular walls.

Eggs, peanuts, and beans are representative foods causing allergies, but they are very important to food industries due to the excellent nutritional values thereof. Thus, it is not easy to restrict or exclude the intake of those these foods to prevent allergic reactions. Therefore, for the improvement of national health and the development of food industries, the development of low antigenic food materials, which exhibit low allergenicity and retain excellent inherent nutritional values intact, is needed.

The above information disclosed in this Background section is only for enhancement of understanding of the background of the inventive concept, and, therefore, it may contain information that does not form the prior art that is already known in this country to a person of ordinary skill in the art.

### SUMMARY

Exemplary embodiments provide modified glycan structures of glycoproteins (removal of particular sugars) to reduce antigenicity of allergenic foods.

Additional aspects will be set forth in the detailed description which follows, and, in part, will be apparent from the disclosure, or may be learned by practice of the inventive concept.

An exemplary embodiment discloses a method for preparing a low antigenic food, the method including removing a sugar linked to a glycoprotein of an allergenic food.

An exemplary embodiment also discloses a method for preparing a low antigenic glycoprotein, the method including removing a sugar linked to a glycoprotein selected from the group consisting of ovalbumin, ovomucoid, ovotransferrin, β-conglycinin, Ara h1, and Ara h2.

An exemplary embodiment also discloses a low antigenic food composition including the low antigenic glycoprotein prepared by removing a sugar linked to a glycoprotein selected from the group consisting of ovalbumin, ovomucoid, ovotransferrin, β-conglycinin, Ara h1, and Ara h2 as an active ingredient.

An exemplary embodiment also discloses a method for preparing a low antigenic glycoprotein, the method including removing a glucose linked to a glycoprotein selected from the group consisting of ovalbumin, ovomucoid, ovotransferrin, β-conglycinin, Ara h1, and Ara h2.

An exemplary embodiment also discloses a low antigenic glycoprotein prepared by removing a glucose linked to a glycoprotein selected from the group consisting of ovalbumin, ovomucoid, ovotransferrin, β-conglycinin, Ara h1, and Ara h2.

An exemplary embodiment also discloses a low antigenic food composition including the low antigenic glycoprotein prepared by removing a glucose linked to a glycoprotein selected from the group consisting of ovalbumin, ovomucoid, ovotransferrin, β-conglycinin, Ara h1, and Ara h2 as an active ingredient.

An exemplary embodiment also discloses a low antigenic cosmetic composition including the low antigenic glycoprotein prepared by removing a glucose linked to a glycoprotein selected from the group consisting of ovalbumin, ovomucoid, ovotransferrin, β-conglycinin, Ara h1, and Ara h2 as an active ingredient.

The foregoing general description and the following detailed description are exemplary and explanatory and are intended to provide further explanation of the claimed subject matter.

### BRIEF DESCRIPTION OF THE DRAWINGS

The accompanying drawings, which are included to provide a further understanding of the inventive concept, and are incorporated in and constitute a part of this specification, illustrate exemplary embodiments of the inventive concept, and, together with the description, serve to explain principles of the inventive concept

FIG. 1 depicts analysis results of component sugars of ovalbumin (OVA) (Fuc: fucose, Ara: arabinose, Gal: galactose, Man: mannose, Xyl: xylose, GalN: galactosamine, GlcN: glucosamine, Neu5Ac: N-acetylneuraminic acid, Neu5Gc: (N-glycolylneuraminic acid).

FIG. 2 depicts graphs showing analysis results of component sugars of ovalbumin (OVA) through HPLC using amide column ((A): 2-aminobenzamide-labeled glucose homopolymer standard graph; (B): N-glycosylation profile of ovalbumin oligosaccharides).

FIG. 3 depicts graphs showing measurement results of molecular weights of ovalbumin oligosaccharides, analyzed by HPLC, through matrix-assisted laser desorption/ionization time-of-flight mass spectrometry (MALDI-TOF-MS) ((A): analysis results of oligosaccharides of 2-aminobenzamide-labeled ovalbumin, (B) analysis results of oligosaccharides of hypermethylated ovalbumin).

FIG. 4 depicts an SDS-PAGE image for verifying protein degradation or denaturation after exoglycosidase treatment (1: molecular marker, 2: untreated ovalbumin (control), 3: mannosidase-treated ovalbumin, 4: galactosidase-treated ovalbumin, 5: N-acetylglucosaminidase-treated ovalbumin).

FIG. 5 depicts graphs showing analysis and comparison results of changes in oligosaccharides of ovalbumin after exoglycosidase treatment ((A): oligosaccharides of untreated ovalbumin, (B): oligosaccharides of mannosidase-treated ovalbumin, (C): oligosaccharides of N-acetylglucosaminidase-treated ovalbumin, (D): oligosaccharides of galactosidase-treated ovalbumin).

FIG. 6 depicts a graph showing measurement results of the total IgE production by the immunization of exoglycosidase-treated ovalbumin (Normal: group in which an immune response is not induced, OVA: group in which an immune response is induced by untreated ovalbumin, G-OVA: group in which an immune response is induced by galactosidase-treated ovalbumin, M-OVA: group in which an immune response is induced by mannosidase-treated ovalbumin, N-OVA: group in which an immune response is induced by N-acetylglucosaminidase-treated ovalbumin).

FIG. 7 depicts graphs showing measurement results of cytokines (IL-4, IL-5, and IL-17) produced by re-stimulating spleen cells of mice, immunized with exoglycosidase-treated ovalbumin, via respective antigens ([X axis] OVA: group in which a primary immune response is induced by untreated ovalbumin, G-OVA: group in which a primary immune response is induced by galactosidase-treated ovalbumin, M-OVA: group in which a primary immune response is induced by mannosidase-treated ovalbumin, N-OVA: group in which a primary immune response is induced by N-acetylglucosaminidase-treated ovalbumin; [examples in Box] Media: group in which re-stimulation is not induced, OVA: group in which re-stimulation is induced by untreated ovalbumin, G-OVA: group in which re-stimulation is induced by galactosidase-treated ovalbumin, M-OVA: group in which re-stimulation is induced by mannosidase-treated ovalbumin, N-OVA: group in which re-stimulation is induced by N-acetylglucosaminidase-treated ovalbumin).

FIG. 8 shows analysis results of component sugars of ovomucoid (OM) (Gal: galactose, Man: mannose, GlcN: glucosamine, NeuAc: N-acetylneuraminic acid).

FIG. 9 depicts graphs showing analysis results of component sugars of ovomucoid (OM) through HPLC using amide column ((A): 2-aminobenzamide-labeled glucose homopolymer standard graph; (B): N-glycosylation profile of ovomucoid oligosaccharides).

FIG. 10 depicts graphs showing measurement results of molecular weights of ovomucoid oligosaccharides, analyzed by HPLC, through matrix-assisted laser desorption/ionization time-of-flight mass spectrometry (MALDI-TOF-MS) ((A): analysis results of oligosaccharides of 2-aminobenzamide-labeled ovomucoid, (B) analysis results of oligosaccharides of hypermethylated ovocomuid).

FIG. 11 depicts graphs showing analysis and comparison results of changes in oligosaccharides of ovomucoid after exoglycosidase treatment ((A): oligosaccharides of untreated ovomucoid, (B): oligosaccharides of galactosidase-treated ovomucoid, (C): oligosaccharides of mannosidase-treated ovomucoid, (D): oligosaccharides of N-acetylglucosaminidase-treated ovomucoid, (E): oligosaccharides of sialidase-treated ovomucoid).

FIG. 12 depicts graphs showing measurement results of the total IgE production by the immunization of exoglycosidase-treated ovomucoid (Nor: group in which an immune response is not induced, OM: group in which an immune response is induced by untreated ovomucoid, G-OM: group in which an immune response is induced by galactosidase-treated ovomucoid, M-OM: group in which an immune response is induced by mannosidase-treated ovomucoid, N-OM: group in which an immune response is induced by N-acetylglucosaminidase-treated ovomucoid, S-OM: group in which an immune response is induced by sialidase-treated ovomucoid).

FIG. 13 depicts graphs showing measurement results of cytokine (IL-4) produced by re-stimulating spleen cells of mice, immunized with exoglycosidase-treated ovomucoid, by respective antigens (Media: group in which restimulation is not induced, OM: group in which restimulation is induced by untreated ovomucoid, G-OM: group in which restimulation is induced by galactosidase-treated ovomucoid, M-OM: group in which restimulation is induced by mannosidase-treated ovomucoid, N-OM: group in which restimulation is induced by N-acetylglucosaminidase-treaed ovomucoid, S-OM: group in which restimulation is induced by sialidase-treated ovomucoid).

### DETAILED DESCRIPTION OF THE ILLUSTRATED EMBODIMENTS

In the following description, for the purposes of explanation, numerous specific details are set forth in order to provide a thorough understanding of various exemplary embodiments. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments.

In the accompanying figures, the size and relative sizes of elements or components, may be exaggerated for clarity and descriptive purposes. Also, like reference numerals denote like elements.

For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various elements, these elements should not be limited by these terms. These terms are used to distinguish one element from another element. Thus, a first element discussed below could be termed a second element without departing from the teachings of the present disclosure.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

Hereinafter, the inventive concept will be described in detail.

The inventive concept provides a method for preparing a low antigenic food by removing a sugar linked to a glycoprotein of an allergenic food.

Whole eggs, beans, peanuts, and the like are known as allergenic foods, and the glycoproteins contained therein are known to cause allergies.

The term “whole egg” refers to the whole contents in a bird egg. The whole egg covers both the egg yolk and the egg white. The whole egg includes all kinds of whole eggs regardless of their origin of specific birds. The whole egg may preferably be a whole egg of an edible bird egg, for example, a whole hen egg, a whole quail egg, a whole ostrich egg, or a whole duck egg, and more preferably a whole hen egg.

In addition, the whole egg may be the whole contents of the bird egg, and includes contents that are obtained by separating and purifying some ingredients. In addition, the whole egg of the inventive concept includes all those contents that are subjected to processes such as drying and powdering, while being preferably a whole egg powder. In addition, the whole egg of the inventive concept may be one that is obtained by separating protein ingredients. As the whole egg of the inventive concept, a whole egg that is directly separated from a bird egg may be used, or a commercially marketable whole egg powder or the like may be purchased and used.

As examples of the glycoprotein, ovalbumin and ovomucoid, which are yolk proteins, and ovotransferrin, β-conglycinin, Ara h1, and Ara h2 are known, but are not limited thereto.

The term “egg white” refers to a material surrounding the yellow part of a bird egg. Around 90% of the egg white is composed of water. The egg white includes all kinds of an egg white, regardless of the origin of birds. The egg white may preferably be an egg white of an edible bird egg, for example, a hen egg white, a quail egg white, an ostrich egg white, or a duck egg white, and more preferably a hen egg white. In addition, the egg white may be a whole material surrounding the yellow part of the bird egg, and includes some ingredients that are separated and purified. In addition, the egg white as used herein includes those that are subjected to drying and powdering, and preferably may be an egg white powder. In addition, the egg white of the inventive concept may be a particular protein ingredient of the egg white that is separated, and may preferably be selected from the group consisting of ovalbumin, ovomucoid, and a mixture thereof. As the egg white of the inventive concept, an egg white that is directly separated from a bird egg may be used, or a commercially marketable whole egg powder or the like may be purchased and used.

Examples of the egg white protein may be ovalbumin, ovomucoid, ovotransferrin, lysozyme, and globulin. Among them, ovalbumin (54% of the egg white protein), ovomucoid (11% of the egg white protein), and ovotransferrin are known as strong allergy sources, and these proteins are also present in the form of a glycoprotein. Herein, the egg white protein from which a sugar is to be removed may preferably be ovalbumin or ovomucoid.

Egg white albumin or ovalbumin as used herein is described as ovalbumin.

The ovalbumin and ovomucoid, which are a kind of protein contained in the egg white, are representative proteins that account for about 65% of the egg white protein, and contain 10-25% of sugars. The ovalbumin and ovomucoid are the main antigens causing the allergy of the egg white protein. The ovotransferrin is also a kind of glycoprotein contained among the egg white protein, and called conalbumin. The β-conglycinin is a glycoprotein contained in beans, while Ara h1 and Ara h2 are glycoproteins contained in peanuts.

The ovalbumin and ovomucoid of the inventive concept may be derived from egg whites of all types of birds, regardless of the origin of birds, and may be preferably derived from preferably white eggs of edible bird eggs. The egg white may be, for example, a hen egg white, a quail egg white, an ostrich egg white, or a duck egg white, and more preferably a hen egg white.

In addition, the ovalbumin and ovomucoid of the inventive concept include those that are subjected to processes such as drying and powdering, and preferably may be ovalbumin and ovomucoid powders. As the ovalbumin and ovomucoid of the inventive concept, ones that are directly separated from a bird egg may be used, or a commercially marketable ovalbumin and ovomucoid powders or the like may be purchased and used.

The sugar removed from glycoproteins of allergenic foods may be, but not limited to, mannose, galactose, N-acetylglucosamine, N-acetylneuraminic acid, and xylose, arabinose, more preferably mannose, galactose, N-acetylglucosamine, and N-acetylneuraminic acid, and most preferably N-acetylglucosamine.

Specifically, the sugar removed from the ovalbumin of the inventive concept may include, but is not limited thereto, a sugar at the terminal of a particular structured glycan, and may be at least one sugar of (1), (2), and (3) below:

(1) mannose (Man) at the glycan terminal, having a structure of Man α1-3(Man α1-3(Man α1-6) Man α1-6) Man β1-4 GlcNAc β1-4 GlcNAc or Man α1-2 Man α1-3(Man α1-6(Man β1-3) Man α1-6) Man β1-4 GlcNAc β1-4 GlcNAc;

(2) N-acetylglucosamine (GlcNAc) at the glycan terminal, having a structure of GlcNAc β1-4(Man α1-3) (Man α1-6) Man β1-4 GlcNAc β1-4 GlcNAc or GlcNAc β1-2 Man α1-3(GlcNAc β1-2 Man α1-6) Man β1-4 GlcNAc β1-4 GlcNAc; and

(3) galactose (Gal) at the glycan terminal, having a structure of (Gal β1-4 GlcNAc β1-2) Man α1-3(Man α1-6) Man β1-4 GlcNAc β1-4 GlcNAc.

Specifically, the sugar that is removed from the ovomucoid of the inventive concept may include, but is not limited thereto, a sugar at the terminal of a particular structured glycan, and may be at least one sugar of (1), (2), (3), and (4) below:

(1) mannose (Man) at the glycan terminal, having a structure of Man α1-3 (Man α1-6) Man β1-4 GlcNAc β1-4 GlcNAc;

(2) N-acetylglucosamine (GlcNAc) at the glycan terminal, having a structure of GlcNAc β1-4 (GlcNAc β1-2 (GlcNAc β1-4) Man α1-3) (GlcNAc β1-2 Man α1-6) Man β1-4 GlcNAc β1-4 GlcNAc or GlcNAc β1-4 (GlcNAc β1-2 (GlcNAc β1-4) Man α1-3) (GlcNAc β1-2 (GlcNAc β1-4) (GlcNAc β1-6) Man α1-6) Man β1-4 GlcNAc β1-4 GlcNAc;

(3) N-acetylneuraminic acid (NeuAc) at the glycan terminal, having a structure of (NeuAc α2-3 or 6 Gal β1-4|GlcNAc β1-4 (GlcNAc β1-2 (GlcNAc β1-4) Man α1-3) (GlcNAc β1-2 (GlcNAc β1-4) Man α1-6) Man β1-4 GlcNAc β1-4 GlcNAc) or (NeuAc α2-3 or 6 Gal β1-4|GlcNAc β1-4 (GlcNAc β1-2 (GlcNAc β1-4) (GlcNAc β1-6) Man α1-3) (GlcNAc β1-2 (GlcNAc β1-4) Man α1-6) Man β1-4 GlcNAc β1-4 GlcNAc); and

(4) galactose (Gal) at the glycan terminal, having a structure of (Gal β1-4 GlcNAc β1-4 (GlcNAc β1-2 (GlcNAc β1-4) Man α1-3) (GlcNAc β1-2 (GlcNAc β1-4) (GlcNAc β1-6) Man α1-6) Man β1-4 GlcNAc β1-4 GlcNAc).

The method of removing a sugar that is linked to a glycoprotein of an allergenic food of the inventive concept is performed by treating the glycoprotein with an exoglycosidase.

Antigenicity refers to an ability to induce an antibody production. A low antigenic glycoprotein of the inventive concept refers to a glycoprotein, of which the probability of antibody production is lower than those of general allergenic glycoproteins, leading to a low probability of causing allergy.

The mixing of an exoglycosidase and an allergenic glycoprotein, specifically, a glycoprotein, which is selected from the group consisting of ovalbumin, ovomucoid, ovotransferrin, β-conglycinin, Ara h1, and Ara h2, results in hydrolyzing sugar residues contained in the glycoprotein, and the removal of the sugar residue from the glycoprotein leads to a reduction in antigenicity of the glycoprotein. The inventive concept presents, for the first time, these findings.

The exoglycosidase may be xylosidase, arabinosidase, mannosidase, galactosidase, N-acetylglucosaminidase, or sialidase, more preferably mannosidase, galactosidase, N-acetylglucosaminidase, or sialidase, and most preferably N-acetylglucosaminidase.

In addition, the exoglycosidases above may be purchased, synthesized, or purified in strains transformed with recombinant vectors. Alternatively, microorganisms which secrete the exoglycosidases above may be directly used. The microorganisms that secrete the exoglycosidases may be Streptococcus pneumonia, Aspergillus oryzae, Pseudomonas fluorescens, Escherichia coli, Kluyveromyces lactis, Bacteroides fragillis, Saccharomyces fragillis, Xanthomonas manihotis, or Aspergillus niger. pneumonias may secrete N-acetylglucosaminidase and galactosidase; A. oryzae and P. fluorescens may secrete N-acetylglucosaminidase; and E. coli, K lactis, B. fragillis, and S. fragillis may secrete galactosidase. In addition, X. manihotis and A. niger secrete mannosidase.

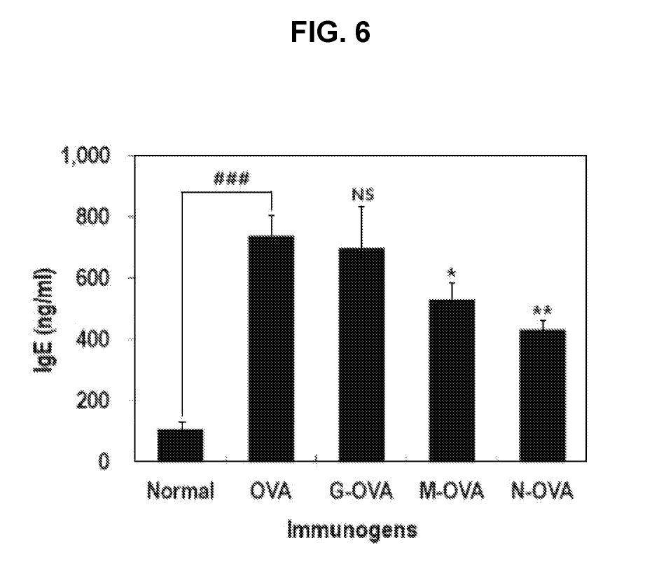

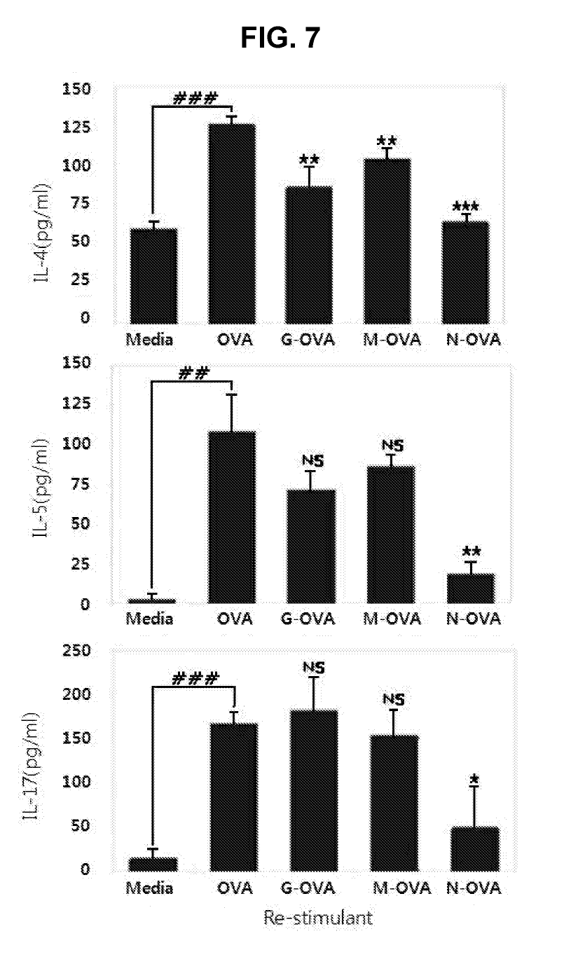

An example of the inventive concept verified that the antigenicity of ovalbumin was reduced through a process in which ovalbumin, among the glycoproteins of the allergenic foods, was treated with mannosidase, galactosidase, and N-acetylglucosaminidase, respectively, to remove mannose, galactose, and GlcNAc linked to the ovalbumin. The increases in IgE, IL-4, IL-5, and IL-17 productions were significantly less in test groups undergoing the sugar removal process rather than the control group receiving ovalbumin treatment only (see Example 4, and FIGS. 6 and 7).

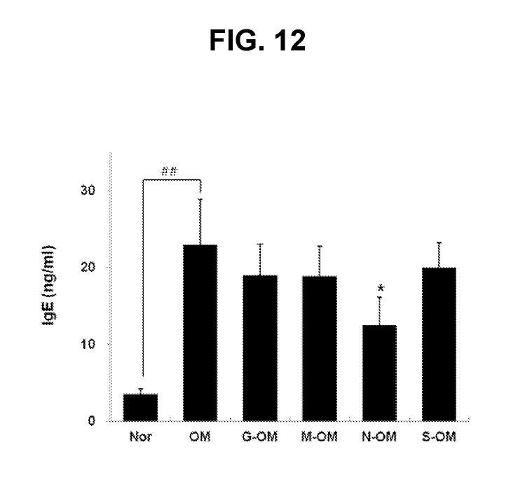

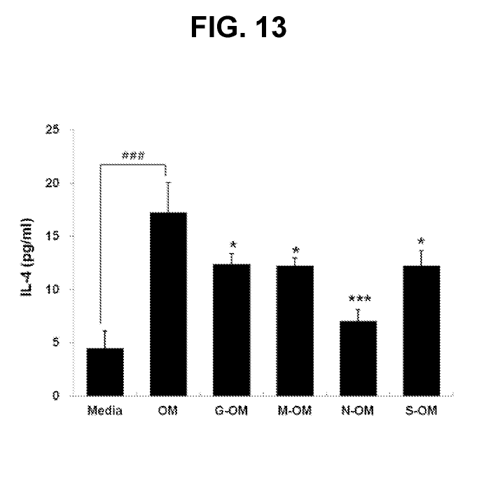

Another example of the inventive concept verified that the antigenicity of ovomucoid was reduced through a process in which ovomucoid was treated with mannosidase, galactosidase, sialidase, and N-acetylglucosaminidase, respectively, to remove mannose, galactose, N-acetylglucosamine, and N-acetylneuraminic acid, linked to the ovomucoid. As a result, the increases in IgE and IL-4 production were significantly less in test groups undergoing the sugar removal process rather than the control group receiving ovomucoid treatment only (see Example 8, and FIGS. 12 and 13).

Therefore, the removal of sugars linked to glycoproteins of allergenic foods can reduce antigenicity of the glycoproteins, and low antigenic foods can be prepared by a method comprising the step of removing a sugar linked to a glycoprotein of an allergenic food. Preferably, low antigenic ovalbumin or low antigenic ovomucoid can be prepared by the method for removing the sugar linked to ovalbumin or ovomucoid.

In another example of the inventive concept, the kind of the glycan structure linked to existing ovalbumin and the glycan structure of low antigenic ovalbumin of the inventive concept were analyzed through HPLC. The results confirmed that mannose (Man) at the glycan terminal, having a structure of Man α1-3(Man α1-3(Man α1-6) Man α1-6) Man β1-4 GlcNAc β1-4 GlcNAc or Man α1-2 Man α1-3(Man α1-6(Man β1-3) Man α1-6) Man β1-4 GlcNAc β1-4 GlcNAc; or N-acetylglucosamine (GlcNAc) at the glycan terminal, having a structure of GlcNAc β1-4(Man α1-3) (Man α1-6) Man β1-4 GlcNAc β1-4 GlcNAc or GlcNAc β1-2 Man α1-3(GlcNAc β1-2 Man α1-6) Man β1-4 GlcNAc β1-4 GlcNAc; or galactose (Gal) at the glycan terminal, having a structure of (Gal β1-4 GlcNAc β1-2) Man α1-3(Man α1-6) Man β1-4 GlcNAc β1-4 GlcNAc, was removed in the low antigenic ovalbumin of the inventive concept.

In still another example of the inventive concept, the kind of the glycan structure linked to existing ovomucoid and the glycan structure of low antigenic ovomucoid of the inventive concept were analyzed through HPLC. The results confirmed that mannose (Man) at the glycan terminal, having a structure of Man α1-3 (Man α1-6) Man β1-4 GlcNAc β1-4 GlcNAc; N-acetylglucosamine (GlcNAc) at the glycan structure, having a structure of GlcNAc β1-4 (GlcNAc β1-2 (GlcNAc β1-4) Man α1-3) (GlcNAc β1-2 Man α1-6) Man β1-4 GlcNAc β1-4 GlcNAc or GlcNAc β1-4 (GlcNAc β1-2 (GlcNAc β1-4) Man α1-3) (GlcNAc β1-2 (GlcNAc β1-4) (GlcNAc β1-6) Man α1-6) Man β1-4 GlcNAc β1-4 GlcNAc; N-acetylneuraminic acid (NeuAc) at the glycan terminal, having a structure of (NeuAc α2-3 or 6 Gal β1-4 GlcNAc β1-4 (GlcNAc β1-2 (GlcNAc β1-4) Man α1-3) (GlcNAc β1-2 (GlcNAc β1-4) Man α1-6) Man β1-4 GlcNAc β1-4 GlcNAc) or (NeuAc α2-3 or 6 Gal β1-4 GlcNAc β1-4 (GlcNAc β1-2 (GlcNAc β1-4) (GlcNAc β1-6) Man α1-3) (GlcNAc β1-2 (GlcNAc β1-4) Man α1-6) Man β1-4 GlcNAc β1-4 GlcNAc); or galactose (Gal) at the glycan terminal, having a structure of (Gal β1-4 GlcNAc β1-4 (GlcNAc β1-2 (GlcNAc β1-4) Man α1-3) (GlcNAc β1-2 (GlcNAc β1-4) (GlcNAc β1-6) Man α1-6) Man β1-4 GlcNAc β1-4 GlcNAc), was removed in the low antigenic ovomucoid of the inventive concept.

The sugar-deficient glycoprotein of the inventive concept is characterized by being deficient in at least one sugar linked to the terminal of the glycoprotein, and thus having reduced antigenicity to cause allergy. The inventive concept presents, for the first time, this glycoprotein with a particular structure in which the terminal sugar of the glycan is removed.

The low antigenic glycoprotein of the inventive concept can be prepared by known glycotechnologies, such as treatment with various glycolytic enzymes.

In addition, the inventive concept provides a method for preparing a low antigenic glycoprotein, the method comprising a step of removing a sugar linked to a glycoprotein selected from the group consisting of ovalbumin, ovomucoid, ovotransferrin, β-conglycinin, Ara h1, and Ara h2.

In addition, the inventive concept provides a low antigenic glycoprotein prepared by the method.

The low antigenic glycoprotein of the inventive concept is characterized by being prepared by the method for preparing a low antigenic glycoprotein of the inventive concept. The low antigenic glycoprotein of the inventive concept includes those that are subjected to processes such as drying and powdering, preferably glycoprotein powders, more preferably ovalbumin or ovomucoid powders.

The low antigenic glycoprotein of the inventive concept causes no allergies due to an extremely low antigenicity thereof, and retains its excellent nutritional values intact, and thus can be used as a raw material for preparing a food or cosmetic composition.

Therefore, the inventive concept provides a low antigenic food composition comprising the low antigenic glycoprotein of the inventive concept as an active ingredient.

The food composition of the inventive concept includes compositions in all types including functional foods, nutritional supplements, health foods, and food additives. The above types of food composition may be prepared in various forms by general methods known in the art.

For example, for the health foods, the low antigenic glycoprotein of the inventive concept is prepared in the form of tea, juice, and drink for drinking thereof, or granulized, capsulated, or powdered for the intake thereof.

In addition, the functional food may be prepared by adding the antigenic glycoprotein of the inventive concept to beverages (including fermented beverages and alcoholic beverages), fish, meat and its processed foods, breads and noodles, confectionery, dairy products, retort foods, frozen foods, and the like.

Further, in order to use the antigenic glycoprotein of the inventive concept in the form of a food additive, the antigenic glycoprotein may be prepared in the form of a powder or concentrate.

In cases where the antigenic glycoprotein of the inventive concept is used for a food composition, the antigenic glycoprotein may be added per se or used together with other food ingredients; may be appropriately used by general methods; and may be used together with a physiologically acceptable sugar, an organic acid, and an organic polymer. The sugar may include refined sugars, mannitol, glucose, sorbitol, xylitol, inositol, lactose, and fructose. The organic acid may include citric acid, ascorbic acid, and amino acids. The mixing amount of the low antigenic glycol protein of the inventive concept may be appropriately determined according to its presumed purpose (the prevention of allergy, the promotion of health, or a therapeutic treatment). In general, the low antigenic glycoprotein may be added in a content of 0.01-100 weight %, and preferably 20-95 weight % on the basis of the raw materials for food preparation. In general, when the composition is taken for a long period of time for the purpose of health and sanitation or health control at the time of food or beverage preparation, the content of the low antigenic glycoprotein may be below the above ranges. Since the active ingredient is not problematic in regards to safety, the content of the active ingredient may exceed the above described ranges.

In addition, the inventive concept provides a low antigenic cosmetic composition comprising the low antigenic glycoprotein of the inventive concept as an active ingredient.

The cosmetic composition of the inventive concept may contain the low antigenic glycoprotein in a content of 0.01-50 weight % on the basis of the total weight of the composition. The cosmetic composition may use the low antigenic glycoprotein of the inventive concept per se or in a diluted form as needed.

The cosmetic composition of the inventive concept may be prepared in a liquid or solid form by using a base material, an adjuvant, and an additive that are generally used in the cosmetic field. Examples of the liquid or solid cosmetics may include, but are not limited to, tonics, creams, lotions, and bath preparations. The base material, adjuvant, and additive that are generally used in the cosmetic field are not particularly limited to, and may include, for example, water, alcohols, propylene glycol, stearic acid, glycerol, cetyl alcohol, and liquid paraffin.

In another example of the inventive concept, it was checked whether proteolysis occurred by the enzyme by measuring the molecular weight of the protein of the enzyme-treated white egg. The results verified that proteolysis did not occur in cases where the preparing method of the inventive concept was employed (see example 3-2). Therefore, a low antigenic glycoprotein, which maintains a nutritional value due to the absence of proteolysis regardless of the reduced antigenicity, can be prepared by the method of the inventive concept.

As set forth above, the inventive concept established a method for reducing antigenicity of a glycoprotein of an allergenic food by removing a sugar linked to the glycoprotein, and thus, a low antigenic glycoprotein can be prepared. Therefore, the glycoprotein of the inventive concept has a very low probability of causing allergy, and maintains inherent nutritional values intact, and thus the inventive concept is effective in the preparation of high-nutritional foods and cosmetics.

Hereinafter, the inventive concept will be described in detail with reference to the following examples.

However, the following examples are merely for illustrating the inventive concept and are not intended to limit the scope of the inventive concept.

### Example 1

### Analysis of Component Sugars of Ovalbumin (OVA)

4 N HCl was added to 1 mg OVA (Sigma-Aldrich, USA), followed by reaction at 100° C. for 4 hours. The sample cooled at room temperature was dried using Speed-vac, and the drying process was twice repeated using purified water. The sample was dissolved in 50 μL of purified water, and then analyzed by HPAEC-PAD (ICS-3000, Dionex, USA) using CarboPac PA1 (Dionex, USA) column with mobile phase A: 200 mM NaOH and B: D.W. at 0.5 mL/min for 80 minutes.

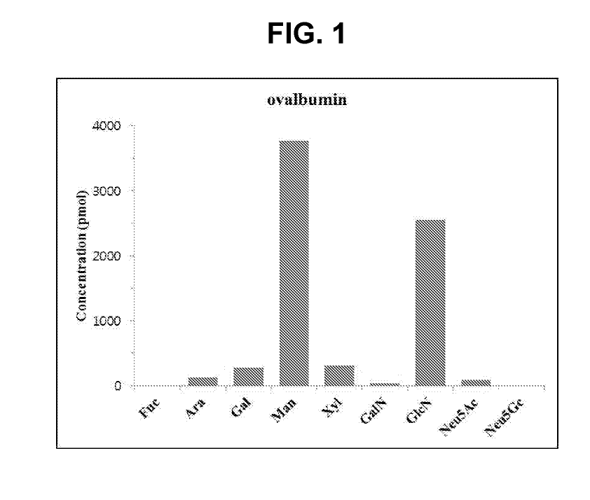

As a result of analyzing component sugars of OVA, it can be seen from FIG. 1 that mannose and N-acetylglucosamine are the largest proportion in OVA, while small amounts of xylose, galactose, and arabinose are present in OVA.

### Example 2

### Analysis of Oligosaccharides of OVA

<2-1> Separation of Oligosaccharides by Enzyme Treatment

1.5 mg of OVA was dissolved in 0.01 M Tris-HCl (pH 8.0), and then heated at 100° C. for 2 minutes for denaturation. 10 μL of trypsin (1 mg/mL, Milli Q water), 10 μL of chymotrypsin (1 mg/mL, Milli Q water), and 1 μL of 1 M CaCl₂ were added, and then a reaction was conducted at 37° C. to make peptides, followed by drying. 30 μL of 0.5 M citrate-phosphate buffer (pH 5.0) was added to the dried sample, and 10 μL (1 mU/100 μL) of glycoamidase A was added, and then a reaction was conducted at 37° C., to separate sugars from the peptides, followed by drying. 50 μL of 1 M Tris-HCl (pH 8.0) was added to the dried sample, and 10 of pronase (1 mg/mL, Milli Q water) was added, followed by reaction at 37° C., thereby hydrolyzing the peptides into amino acids.

<2-2> Analysis of Oligosaccharides by 2-Aminobenzamide (2-AB) Labeling and HPLC

Only oligosaccharides were separated from the sample after the completion of the 3-stage enzyme reaction through Carbograph columns (SPE Carbo, GRACE), followed by drying. 1 mg of the dried sample was dissolved in 20 μL of 2-AB labeling solution (5 mg anthranilamide, 6 mg sodium cyanoborohydride, 70 μL DMSO, 30 μL acetic acid), followed by reaction at 65° C. for 3 hours. After the completion of the reaction, the reagent was removed through cellulose column (cellulose C6413, Sigma-Aldrich, USA), and the reaction material was filtered using 0.45 um filter syringe. 20 μg of the sample was taken, and then dissolved in 20 μL of solvent A (50 mM ammonium formate, pH 4.4) and 80 μL of solvent B (acetonitrile, ACN), and 50 μL (10 μg) of the solution was injected into to HPLC. Here, the sample was analyzed using the TSK-gel amide 80 column (sigma, USA) with mobile phases A and B at 0.4-1 mL/min for 160 minutes.

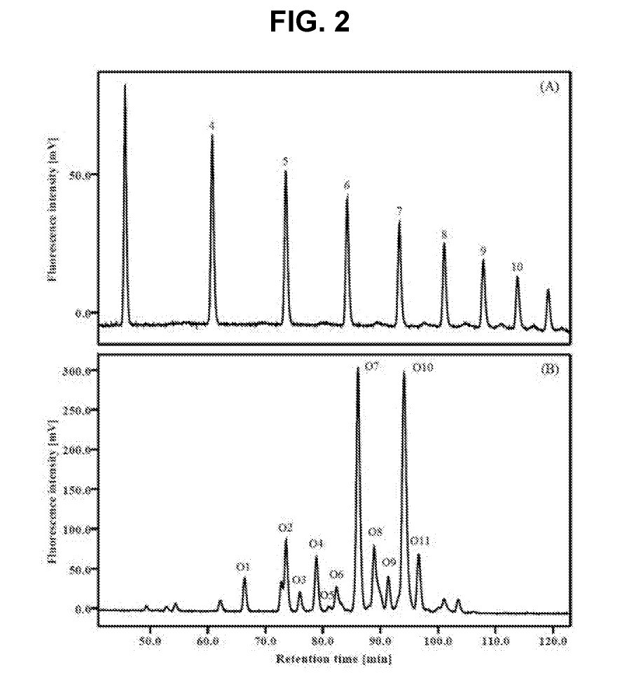

As a result of HPLC analysis of OVA using amide column (TSK-GEL Amide-80, TOSOH, Japan), it can be seen from FIG. 2 that 11 peaks could be confirmed (B), and of these, O7 and O10 had very high intensities, and thus these two peaks are the most abundant in the oligosaccharides of OVA.

<2-3> Analysis of Oligosaccharides by Permethylation and MALDI-TOF Mass

Only oligosaccharides were separated from the sample after the completion of the 3-stage enzyme reaction through Carbograph columns (SPE Carbo, GRACE, USA), followed by drying. 50 μg of the dried sample was dissolved in the permethylation solution (90 μL DMSO, 2.7 μL Milli Q water, 35 μL iodomethane). The solution was allowed to flow through the micro spin column filled with NaOH beads, followed by centrifugation at 400×g for 1 minute. The flow through was collected, and again put on the micro spin column, followed by centrifugation at 400×g for 1 minute, and this procedure was repeated eight times. Last, 150 μL of ACN was put on the micro spin column, followed by centrifugation at 400×g for 1 minute, and thus the flow through was all collected. 400 μL of chloroform and 1 mL of 500 mM NaCl were added to the collected flow through, followed by shaking, and then the solution was centrifuged at 200×g for 1 minute to remove the supernatant. 1 mL of 500 mM NaCl was again added, followed by shaking and then centrifugation, and then the lower liquid was taken. The taken liquid was dried, and the dried material was dissolved in 4 μL of 50% methanol, and mixed with DHB matrix at a ratio of 1:1. The mixture was loaded and sufficiently dried on MALDI-TOF plate (Bruker Daltonics, Germany), and then the mass thereof was measured (ULTRAflex III, Bruker Daltonics, Germany).

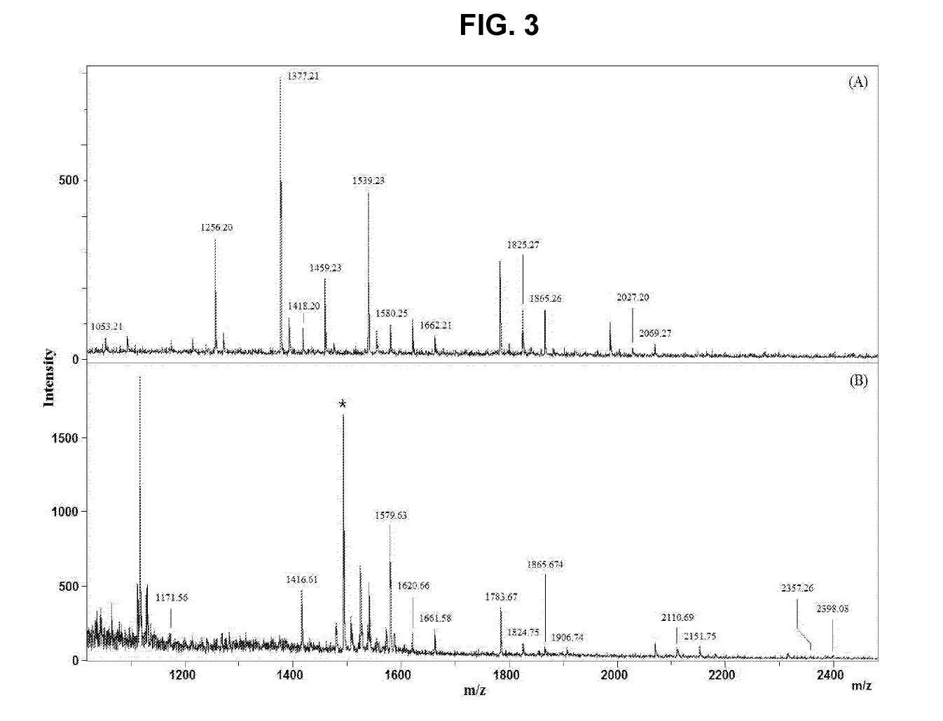

As a result of determining molecular weights of fixed amounts of 2-aminobenzamide-labeled OVA and permethylated OVA using MALDI-TOF MS (see FIG. 3), most molecular weights corresponding to GU values obtained through HPLC (TSK-gel amide 80 column) could be determined. Through HPLC and MALDI-TOF MS, structures of the corresponding oligosaccharides could be confirmed, and high-mannose oligosaccharides were confirmed to be O7 and O10. O7 and O10 accounted for 27% and 28% of the total oligosaccharides, respectively. Besides the high-mannose oligosaccharide, the existences of Manα1-3(Manα1-6)Manβ1-4GlcNAcβ1-4GlcNAc, which is the core structure of an oligosaccharide, and complex, hydride type oligosaccharides were confirmed. Bi- and tetraantennary oligosaccharides were confirmed, and of these, some oligosaccharides were confirmed to include galactose (see Table 1).

TABLE 1
N-Glycan of Oligosaccharides of AB-Labeled and Permethylated OVA

| | | | | MW | | | | | |
|---|---|---|---|---|---|---|---|---|---|
| | Retention | | | observed | | reported | | | Inten- |
| Peak | time | GU | | AB | PerMe | AB | PerMe | | sity |
| No. | (min) | observed | reported | [M + Na] | [M + Na] | [M + Na] | [M + Na] | structure | (%) |
| O1 | 66.4 | 4.42 | 4.4 | 1053.206 | 1171.563 | 1053.3965 | 1171.5834 | 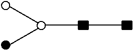 | 3.7 |
| O2 | 73.6 | 5.00 | 4.97 | 1256.195 | 1416.61 | 1256.4759 | 14167098 | 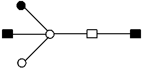 | 7.8 |
| O3 | 76.0 | 5.21 | 5.31 | 1459.233 | 1661.579 | 1459.5553 | 1661.8362 | 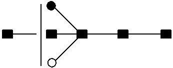 | 2.2 |
| O4 | 78.9 | 5.48 | 5.45 | 1459.233 | 1661.6 | 1459.5553 | 1661.8362 | 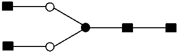 | 6.1 |
| O5 | 81.1 | 5.69 | 5.66 | 1418.202 | 1620.655 | 1418.5287 | 1620.8096 | 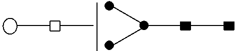 | 0.6 |
| O6 | 82.4 | 5.82 | 5.77 | 1662.205 | 1906.739 | 1662.6347 | 1906.9626 | 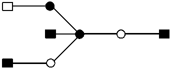 | 2.7 |
| O7 | 86.1 | 6.20 | 6.19 | 1377.209 | 1579.627 | 1377.5021 | 1579.7830 | 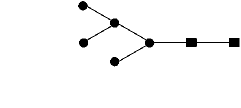 | 27.0 |
| O8 | 88.9 | 6.50 | | 1621.222 | 1865.6 | 1621.6081 | 1865.936 | 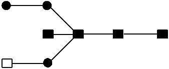 | 7.7 |
| O9 | 91.4 | 6.78 | 6.74 | 2069.274 | 2398.083 | 2068.7248 | 2397.2262 | 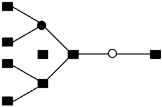 | 4.2 |
| O10 | 94.1 | 7.10 | 7.06 | 1539.228 | 1783.672 | 1539.5549 | 1783.8828 | 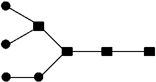 | 28.0 |
| O11 | 96.6 | 7.41 | 7.3 | 2027.202 | 2357.263 | 2027.7669 | 2356.1888 | 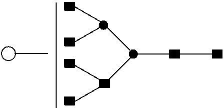 | 6.9 |

( ▪: N-acetylglucosamine,
: mannose,
∘: galactose,
⋆: xylose )

### Example 3

### Modification of Glycan Structure of OVA

<3-1> Modification of Glycan Structure of OVA by Exoglycosidase Treatment

For the modification of the structure of the glycan linked to OVA, exoglycosidase treatment was conducted. Specifically, mannosidase for mannose hydrolysis, galactosidase for galactose hydrolysis, and N-acetylglucosaminidase for GlcNAc hydrolysis were used. For the mannosidase reaction, 482 μL of 50 mM sodium acetate (pH 4.5) was added to 12 mg of OVA, and 2.5 U/18 μL mannosidase was added, followed by overnight incubation at 37° C. For the galactosidase reaction, 497.5 μL of 25 mM sodium phosphate (pH 7.1) was added to 12 mg of OVA, and 2.5 U/2.5 μL galactosidase was added, followed by overnight incubation at 37° C. For the N-acetylglucosaminidase reaction, 460 μL of 50 mM sodium acetate (pH 6.5) was added to 12 mg of OVA, and 2.5 U/40 μL N-acetylglucosaminidase was added, followed by overnight incubation at 37° C. After the completion of each reaction, the enzyme in the reaction liquid was completely removed by ultrafiltration using a 100 kDa molecular weight cut-off membrane, thereby obtaining exoglycosidase derivatives (G-OVA, M-OVA, and N-OVA), of which glycans were modified by removing mannose, galactose, and GlcNAc from OVA, respectively.

<3-2> Molecular Weight Measurement

In order to verify that exoglycosidases, such as mannosidase, galactosidase, and N-acetylglucosaminidase, do not cleave proteins per se when they cleave off glycan residues of OVA, the change in the molecular weight of OVA and derivatives thereof was investigated using SDS-PAGE.

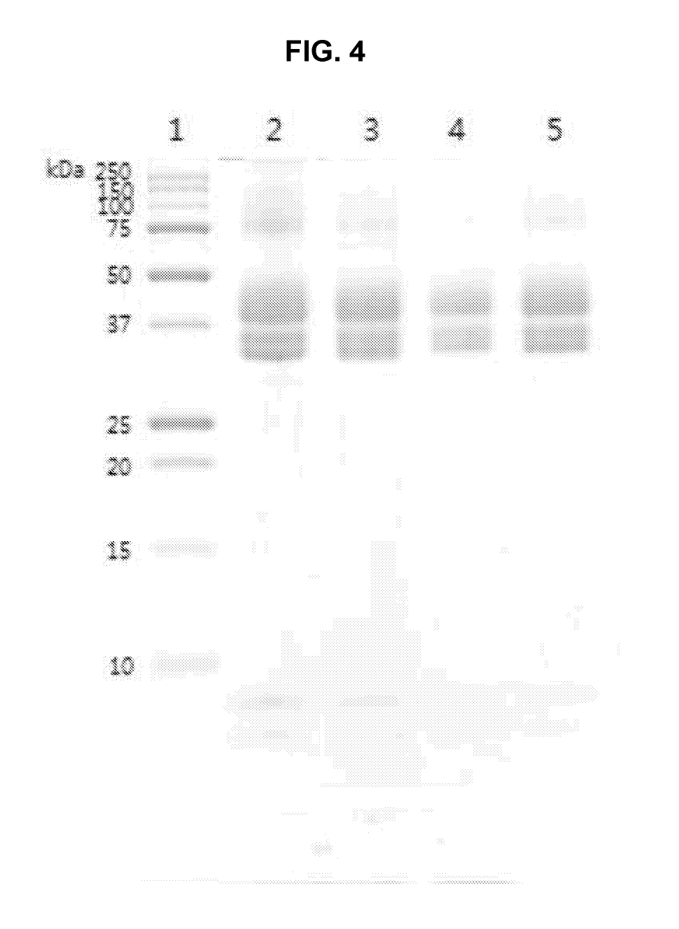

The results confirmed that there was little change in the molecular weight (see FIG. 4), and thus it was confirmed that exoglycosidase treatment did not cause protein hydrolysis or denaturation.

<3-3> HPLC Analysis

The oligosaccharide changes of OVA, in which the terminal portions of the oligosaccharides were cleaved off using exoglycosidases by the same method as in example <2-2>, were analyzed through HPLC.

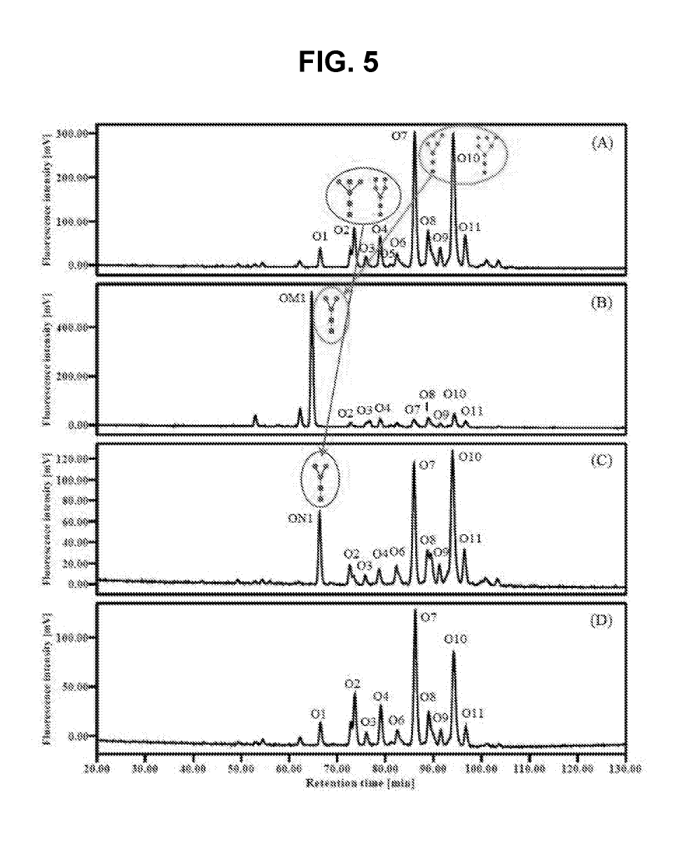

As a result, as shown in FIG. 5, in the case of a derivative prepared by treating OVA with mannosidase (hereinafter, M-OVA), Man α1-3(Man α1-3(Man α1-6) Man α1-6) Man β1-4 GlcNAc β1-4 GlcNAc corresponding to O7 peak and Man α1-2 Man α1-3(Man α1-6(Man β1-3) Man α1-6) Man β1-4 GlcNAc β1-4 GlcNAc corresponding to O10 peak were significantly reduced in the chromatogram of (B), and the significantly increased OM1 peak (Man α1-3(Man α1-6) Man β1-4 GlcNAc β1-4 GlcNAc) was confirmed through comparison of intensity (%) between (A) and (B) of Table 2.

In the case of a derivative prepared by treating OVA with N-acetylglucosaminidase (hereinafter, N-OVA), GlcNAc β1-4(Man α1-3) (Man α1-6) Man β1-4 GlcNAc β1-4 GlcNAc corresponding to O2 peak and GlcNAc β1-2 Man α1-3(GlcNAc β1-2 Man α1-6) Man β1-4 GlcNAc β1-4 GlcNAc corresponding to O4 peak were reduced in the chromatogram of (C), and the significantly increased ON1 peak (Man α1-3(Man α1-6) Man β1-4 GlcNAc β1-4 GlcNAc) was confirmed through comparison of intensity (%) between (A) and (C) of Table 2. It was verified from the chromatogram of (C) that the glycans of high mannose oligosaccharides O7 and O10 were not cleaved.

In the case of a derivative prepared by treating OVA with galactosidase (hereinafter, G-OVA), (Gal β1-4 GlcNAc β1-2) Man α1-3(Man α1-6) Man β1-4 GlcNAc β1-4 GlcNAc corresponding to 05 peak was predicted to be reduced, and was confirmed to be very less so that it was not detected (ND), through the intensity (%) of (A) in Table 2. In addition, it was verified through the chromatogram of (D) and comparison of intensity (%) between (A) and (D) in Table 2 that, besides 05 having galactose linked to a terminal portion of the oligosaccharide, 011 was reduced from 6.9% to 4.0%.

TABLE 2
Intensity Comparison among Oligosaccharides
of OVA and Derivatives Thereof

Intensity(%)

| Peak No. | (A) | (B) | (C) | (D) |
|---|---|---|---|---|
| O1 | 3.7 | 56.9_{(ON1)} | 13.0_{(ON1)} | 4.7 |
| O2 | 7.8 | 1.7 | 5.2 | 10.5 |
| O3 | 2.2 | 2.6 | 1.7 | 2.7 |
| O4 | 6.1 | 3.5 | 2.9 | 8.2 |
| O5 | 0.6 | 0.7 | N/D | N/D |
| O6 | 2.7 | 1.8 | 3.3 | 3.2 |
| O7 | 27.0 | 3.3 | 22.1 | 27.7 |
| O8 | 7.7 | 3.9 | 6.2 | 7.0 |
| O9 | 4.2 | 1.7 | 3.6 | 3.3 |
| O10 | 28.0 | 5.7 | 24.3 | 19.2 |
| O11 | 6.9 | 2.6 | 6.3 | 4.0 |

(A) oligosaccharides of OVA;
(B) oligosaccharides of M-OVA;
(C) oligosaccharides of N-OVA;
(D) oligosaccharides of G-OVA;
N/D—not detected

### Example 4

### Comparative Test of Antigenicity of Glycan-Modified OVA

<4-1> Mouse Immunization and Antiserum Collection

OVA and its exoglycosidase derivatives (G-OVA, M-OVA, and N-OVA) were used as antigens for antibody production, while 6-wk aged BALB/c mice were used for the production of antibodies to the respective antigens. Each antigen (10 μg/mouse) was intraperitoneally injected to five mice for each test group at an interval of two weeks a total of two times. The mice were boost-immunized with OVA (10 μg) as an antigen one week after the final immunization, and the antiserum collection was conducted five days after primary and secondary immunization. The antiserum was stored at −20° C. before the measurement of antibody titer.

<4-2> Measurement of Total IgE Production

OVA has been known as an antigen that causes an immune response with a strong T-helper type (Th2) bias in tests using experimental animal models. B cells influenced by Th2 type cytokines, that is, IL-4 and IL-5, mainly produce IgE antibody, causing type 1 immune hypersensitive response. That is, as for the IgE-mediated immune hypersensitive response, the produced IgE antibody, which reacts with receptors (FcgRIII and FceRI) of mast cells of the tissue, is again stimulated by the same antigen, thereby allowing mast cells to release several inflammation factors, such as histamine, prostagladin, and neucotriens through degranulation, causing an allergic reaction. Therefore, the increase in the production of IgE against antigen is an important indicator to cause an immune hypersensitive response.

The total IgE content against each antigen existing in the serum was measured using a sandwich ELISA kit (BD Biosciences, Franklin Lakes, N.J., USA) for mouse IgE measurement according to the manufacturer's instruction. That is, the coating antibody (anti-IgE) was dispensed into each well of flat-bottomed microtiter plate (Nunc. USA) at 100 μL per well using bicarbonate buffer (pH 9.4), and allowed to adhere to each well at 4° C. for 16 hours. Each well was washed three times with PBS-Tween 20 (0.05%; PBST), blocked using 3% skim milk, and then again washed with PBST. Each of the prepared serums was diluted to 50-fold, and added to each well, followed by reaction at room temperature for 2 hours. After washing, the reaction with the secondary antibody to mouse IgE and the HRP conjugate was conducted. TMB was used as a substrate for HRP action. For colorimetric measurement, the reaction was stopped using 2 N H₂SO₄, and the absorbance was read at 450 nm. The total IgE content in the serum was determined by inserting the measured value into a standard curve using an IgE standard material.

As a result of investigating the effect on IgE production by OVA and the derivatives thereof obtained by the treatment of OVA with exoglycosidases, G-OVA immunization showed a similar tendency in the IgE producing activity compared with the control group of OVA immunization, whereas N-OVA and M-OVA immunizations showed a significantly lower IgE production compared with OVA immunization, and especially, N-OVA immunization showed the lowest value, verifying that allergic antigenicity to promote the IgE production was mostly lost (see FIG. 6). This result means that the glycan portion of OVA plays an important role in the IgE production in association with the allergenic antigenicity of OVA, and the allergic antigenicity of OVA can be regulated by glycan control.

<4-3> Tests for OVA Stimulation and Cytokine Measurement and Stimulation on Lymphocytes

The functions of effector B cells involved in humoral immunity are controlled by cytokines having a Th1 bias, such as IL-2, IFN-γ, and GM-CSF, produced by Th1 cells or by IL-4, IL-5, IL-6, and IL-10, produced by Th2 type helper T cells. IL-17, which is a cytokine also produced by Th17 cells, is known as a cytokine that is directly involved in the induction of immune hypersensitivity diseases, such as allergic symptoms, for example, atopic dermatitis, rhinitis, and asthma, and immune hypersensitivity diseases, for example, arthritis and psoriasis, by mainly mediating inflammation-related diseases. These cytokines are involved in the differentiation and proliferation of B cells into effector cells producing several antibodies against respective antigens. That is, the production of IgE antibody to antigens entering the body may be mainly illustrated by cytokines (IL-4, IL-5, IL-6, and IL-10) produced by Th2 cells, and the inflammation inducing function may be illustrated by the production of IL-17, which is a cytokine produced by Th17 cells.

The spleen was taken from the immunized mouse or normal mouse, and the cell density was adjusted to 3×10⁶ cells/well, and dispensed on the 24-well culture plate. OVA, G-OVA, M-OVA, and N-OVA samples with a final antigen concentration of 10 μg/mL were added to each well in which the spleen cells were dispensed, and the cells were cultured at 37° C. in a 5% CO₂ incubator for 72 hours. After the completion of culturing, the amounts of OVA-specific cytokines existing in the culture supernatant were investigated using an ELISA kit (BD Biosciences) for respective cytokines.

As a result of measuring the production of cytokines produced after the spleen cells obtained from the mice immunized with OVA and its derivatives were re-stimulated with respective antigens, as shown in FIG. 7, M-OVA and G-OVA immunized mice showed a significantly reduced IL-4 production, but did not show significantly reduced IL-5 and IL-17 productions, as compared with the control group of OVA immunized mice. Meanwhile, N-OVA showed significantly reduced productions in all the cytokines, as compared with OVA. These results were the same as the results that the N-OVA immunization showed a significantly reduced IgE production as compared with OVA, M-OVA, and G-OVA immunization. Therefore, it is determined that N-OVA derived from OVA is modified to have a structure causing little OVA allergy and inflammation while inhibiting the type 1 immune hypersensitivity response.

### Example 5

### Analysis of Component Sugars of Ovomucoid (OM)

4 N HCl was added to 1 mg OM (Sigma-Aldrich, USA), followed by reaction at 100° C. for 4 hours. The sample cooled at room temperature was dried using a Speed-vac, and then was repeatedly dried twice using purified water. The sample was dissolved in 50 μL of purified water, and then OM having an amount corresponding to 50 pmol was injected. The sample was analyzed by HPAEC-PAD (ICS-3000, Dionex, USA) using CarboPac PA1 (Dionex, USA) column with mobile phases A: 200 mM NaOH and B: D.W. at 0.5 mL/min for 80 minutes. The analysis was repeated three times to obtain the mean and the standard deviation.

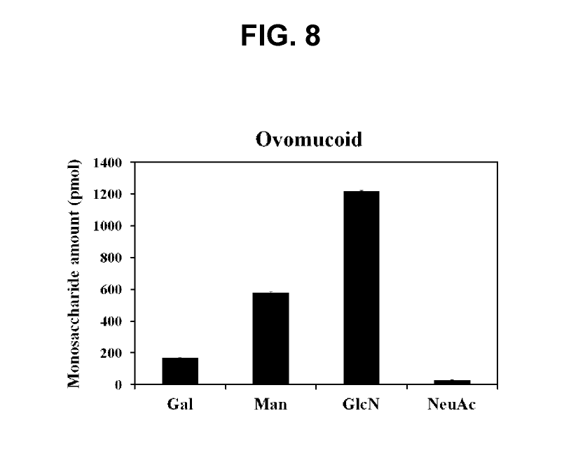

As a result of analyzing component sugars of OM, it can be seen from FIG. 8 that mannose (576.9±6.7 pmol) and N-acetylglucosamine (1219.7±3.0 pmol) are the largest proportion in OM, while small amounts of galactose (167.8±2.8 pmol) and NeuAc (28.7±0.5 pmol) are present in OM.

### Example 6

### Analysis of Oligosaccharides of OM

<6-1> Separation of Oligosaccharides by Enzyme Treatment

1 mg of OM was dissolved in 0.01 M Tris-HCl (pH 8.0), and then heated at 100° C. for 2 minutes for denaturation. 10 μL of trypsin (1 mg/mL, Milli Q water), 10 μL of chymotrypsin (1 mg/mL, Milli Q water), and 1 μL of 1 M CaCl₂ were added, and then a reaction was conducted at 37° C. to make peptides, followed by drying. 30 μL of 0.5 M citrate-phosphate buffer (pH 5.0) was added to the dried sample, and 20 μL (1 mU/100 μL) of glycoamidase A was added, and then a reaction was conducted at 37° C., to separate sugars from the peptides, followed by drying.

<6-2> Analysis of Oligosaccharides by 2-Aminobenzamide (2-AB) Labeling and HPLC

Only oligosaccharides were separated from the sample after the completion of the enzyme reaction in <6-1> through Carbograph columns (SPE Carbo, GRACE), followed by drying. 1 mg of the dried sample was dissolved in 20 μL of 2-AB labeling solution (5 mg anthranilamide, 6 mg sodium cyanoborohydride, 70 μL DMSO, 30 μL acetic acid), followed by reaction at 65° C. for 3 hours. After the completion of the reaction, the reagent was removed through cellulose column (cellulose C6413, Sigma-Aldrich, USA), and the reaction material was filtered using 0.45 um filter syringe. 4 μg of the sample was taken, and then dissolved in 20 μL of solvent A (50 mM ammonium formate, pH 4.4) and 80 μL of solvent B (acetonitrile, ACN), and 50 μL (2 μg) of the solution was injected into to HPLC. Here, the sample was analyzed using TSK-gel amide 80 column (sigma, USA) with mobile phases A and B at 0.4-1 mL/min for 160 minutes.

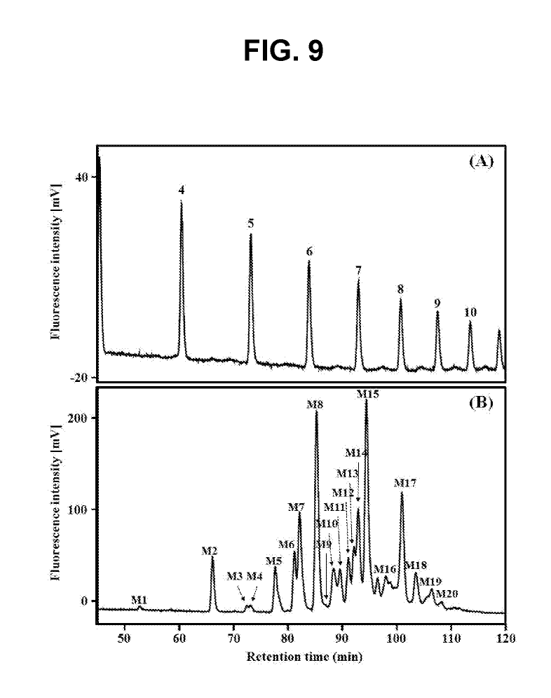

As a result of HPLC analysis of OM using amide column (TSK-GEL Amide-80, TOSOH, Japan), it can be seen from FIG. 9 that 20 separated peaks could be confirmed (B). Glucose unit (GU) values having respective peaks were obtained by comparing the retention times of the peaks with that of the glucose homopolymer standard [Table 3]. Structures thereof were predicted by comparing the GU values thereof with those of oligosaccharides reported on the Glycobase (http://glycobase.nibrtie/glycobase/show_nibrt.action).

<6-3> Analysis of Oligosaccharides by Permethylation and MALDI-TOF Mass

Only oligosaccharides were separated from the sample after the completion of the enzyme reaction in <6-1> through Carbograph columns (SPE Carbo, GRACE, USA), followed by drying. 50 μg of the dried sample was dissolved in the permethylation solution (90 DMSO, 2.7 μL Milli Q water, 35 μL iodomethane). The solution was allowed to flow through the micro spin column filled with NaOH beads, followed by centrifugation at 400×g for 1 minute. The flow through was collected, and again put on the micro spin column, followed by centrifugation at 400×g for 1 minute, and this procedure was repeated eight times. Last, 150 μL of ACN was put on the micro spin column, followed by centrifugation at 400×g for 1 minute, and thus the flow through was all collected. 400 μL of chloroform and 1 mL of 500 mM NaCl were added to the collected flow through, followed by shaking, and then the solution was centrifuged at 200×g for 1 minute to remove the supernatant. 1 mL of 500 mM NaCl was again added, followed by shaking and then centrifugation, and then the lower liquid was taken. The taken liquid was dried, and the dried material was dissolved in 4 μL of 50% methanol, and mixed with DHB matrix at a ratio of 1:1. The mixture was loaded and sufficiently dried on MALDI-TOF plate (Bruker Daltonics, Germany), and then the mass thereof was measured (ULTRAflex III, Bruker Daltonics, Germany).

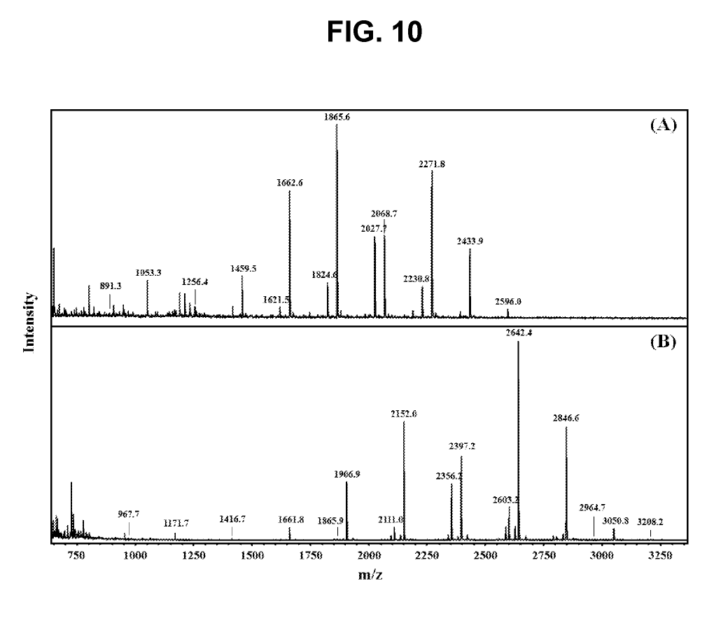

As a result of determining molecular weights of fixed amounts of 2-aminobenzamide-labeled OM and permethylated OM using MALDI-TOF MS (see FIG. 10), most molecular weights corresponding to GU values obtained through HPLC (TSK-gel amide 80 column) could be determined. As a result, it was confirmed that only complex type oligosaccharides were present. Mono- and penta-antennary oligosaccharides were confirmed, and of these, some oligosaccharides were confirmed to include galactose and NeuAc (see Table 3). The structures of M13, M14, M15, M17, M18, M19, and M20 have not yet been reported in the Glycobase, and the structures were predicted using the molecular weight thereof and patterns obtained by exoglycosidase treatment.

TABLE 3

| | | MALDI-TOF MS [M + Na]+ | | | | | | Relative |
|---|---|---|---|---|---|---|---|---|
| | | calculated | | detected | | GU | | quantity |
| Peak | structure | 2-AB | PerMe | 2-AB | PerMe | reported | obserbed | (%) |
| M1 | 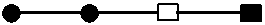 | 891.3 | 967.5 | 891.3 | 967.7 | 3.46 | 3.48 | 0.2 |
| M2 | 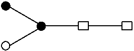 | 1053.4 | 1171.6 | 1053.3 | 1171.7 | 4.40 | 4.44 | 3.0 |
| M3 | 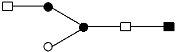 | 1256.5 | 1416.7 | 1256.4 | 1416.7 | 4.93 | 4.95 | 0.4 |
| M4 | 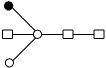 | 1265.5 | 1416.7 | 1256.4 | 1416.7 | 4.97 | 5.01 | 0.4 |
| M5 | 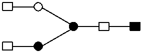 | 1459.5 | 1661.8 | 1459.5 | 1661.8 | 5.45 | 5.42 | 3.3 |
| M6 | 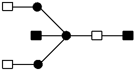 | 1662.6 | 1907.0 | 1662.6 | 1906.9 | 5.76 | 5.76 | 4.1 |
| M7 | 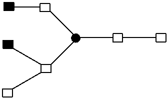 | 1662.6 | 1907.0 | 1662.6 | 1906.9 | 5.90 | 5.85 | 7.3 |
| M8 | 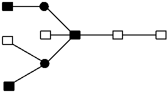 | 1865.7 | 2152.1 | 1865.6 | 2152.0 | 6.14 | 6.17 | 16.6 |
| M9 | 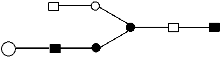 | 1621.6 | 1865.0 | 1621.5 | 1865.9 | 6.38 | 6.34 | 0.5 |
| M10 | 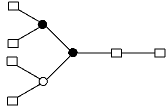 | 1865.7 | 2152.1 | 1865.6 | 2152.0 | 6.53 | 6.51 | 3.7 |
| M11 | 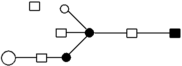 | 1824.7 | 2111.0 | 1824.6 | 2111.0 | 6.62 | 6.64 | 3.3 |
| M12 | 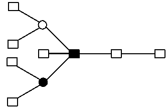 | 2068.8 | 2397.2 | 2068.7 | 2397.2 | 6.74 | 6.82 | 3.8 |
| M13 | 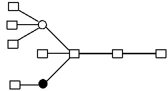 | 2068.8 | 2397.2 | 2068.7 | 2397.2 | —^{a} | 6.94 | 3.2 |
| M14 | 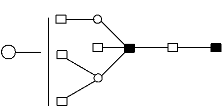 | 2027.8 | 2356.2 | 2027.7 | 2356.2 | —^{a} | 7.03 | 8.3 |
| M15 | 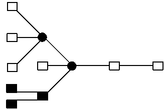 | 2271.9 | 2642.3 | 2271.8 | 2642.4 | —^{a} | 7.21 | 18.5 |
| M16 | 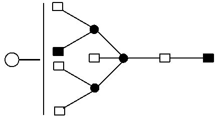 | 2230.8 | 2601.3 | 2230.8 | 2603.2 | 7.64 | 7.67 | 3.2 |
| M17 | 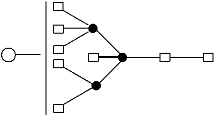 | 2433.9 | 2846.4 | 2433.9 | 2846.6 | —^{a} | 8.07 | 10.5 |
| M18 | 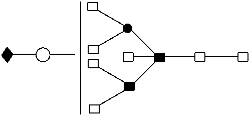 | —^{b} | 2962.5 | nd | 2964.7 | —^{a} | 8.44 | 3.7 |
| M19 | 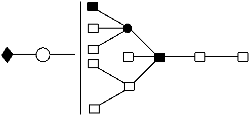 | 2596.0 | 3050.5 | 2596.0 | 3050.8 | —^{a} | 8.86 | 2.9 |
| M20 | 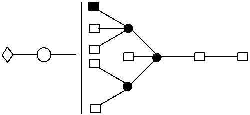 | —^{b} | 3207.6 | nd | 3208.2 | —^{a} | 9.15 | 0.7 |

( ▪: N-acetylglucosamine,
: mannose,
∘: galactose,
♦: xylose )
^{a}structure not yet reported in the Glycobase, predicted by comparing results obtained by exoglycosidase treatment and results of MALDI-TOF.
^{b}structure having NeuAc, not measured in the positive mode, confirmed from permethylated oligosaccharides.
nd: not detected

### Example 7

### Modification of Glycan Structure of OM

<7-1> Modification of Glycan Structure of OM by Exoglycosidase Treatment

For the modification of the structure of the glycan linked to OM, exoglycosidase treatment was conducted. Specifically, galactosidase for galactose hydrolysis, mannosidase for mannose hydrolysis, N-acetylglucosaminidase for GlcNAc hydrolysis, and sialidase for NeuAc hydrolysis were used. For the galactosidase reaction, 997.5 μL of 50 mM sodium phosphate (pH 6.0) was added to 11 mg of OM, and 2.5 U/2.5 μL galactosidase was added, followed by overnight incubation at 37° C. For the mannosidase reaction, 982 μL of 50 mM sodium acetate (pH 4.5) was added to 11 mg of OM, and 2.5 U/18 μL mannosidase was added, followed by overnight incubation at 37° C. For the N-acetylglucosaminidase reaction, 960 μL of 50 mM sodium acetate (pH 6.0) was added to 11 mg of OVA, and 2.5 U/40 μL N-acetylglucosaminidase was added, followed by overnight incubation at 37° C. For the sialidase reaction, 956 μL of 50 mM sodium acetate (pH 6.0) was added to 11 mg of OM, and 44 U sialidase was added, followed by overnight incubation at 37° C. After the completion of each reaction, the enzyme in the reaction liquid was completely removed by ultrafiltration using a 100 kDa molecular weight cut-off membrane, thereby obtaining exoglycosidase derivatives (G-OM, M-OM, N-OM and S-OM), of which glycans were modified by removing mannose, galactose, GlcNAc, and NeuAc from OM, respectively.

<7-2> HPLC Analysis

The oligosaccharide changes of OM, in which the terminal portions of the oligosaccharides were cleaved off using exoglycosidases by the same method as in example <6-2>, were analyzed through HPLC.

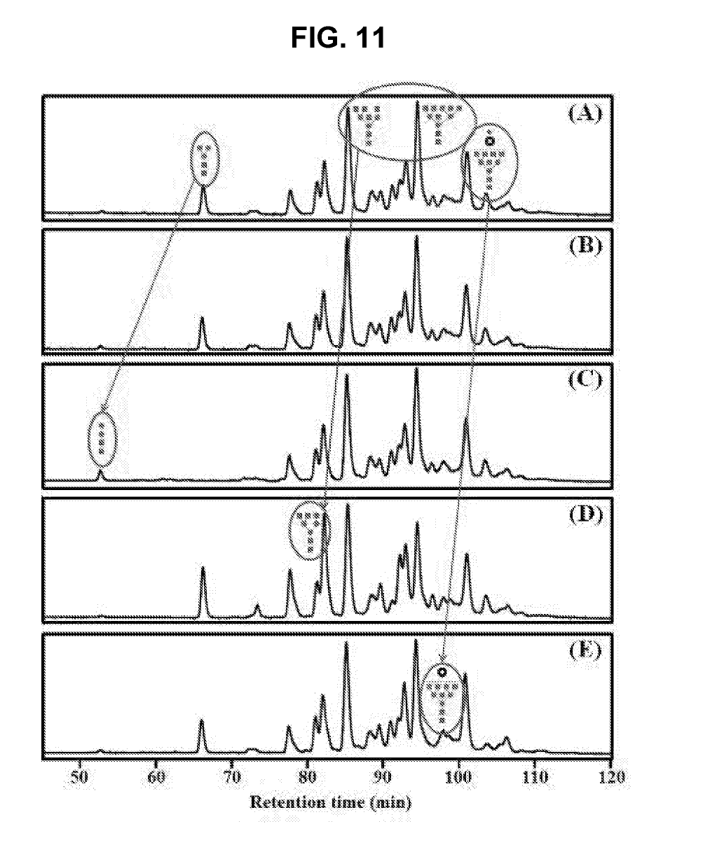

As a result, it was confirmed from FIG. 11 that a derivative prepared by treating OM with galactosidase (hereinafter, G-OM) did not show a large change.

It was confirmed that, in the case of a derivative prepared by treating OM with mannosidase (hereinafter, M-OM), M2 peak having a structure of Man α1-3 (Man α1-6) Man β1-4 GlcNAc β1-4 GlcNAc was absent in the chromatogram of (C), and a peak having a structure of M1 (Man α1-6 Man β1-4 GlcNAc β1-4 GlcNAc) was significantly increased.

In addition, it was confirmed that, in the case of a derivative obtained by treating OM with N-acetylglucosaminidase (hereinafter, N-OM), GlcNAc β1-4 (GlcNAc β1-2 (GlcNAc β1-4) Man α1-3) (GlcNAc β1-2 Man α1-6) Man β1-4 GlcNAc β1-4 GlcNAc corresponding to M8 and GlcNAc β1-4 (GlcNAc β1-2 (GlcNAc β1-4) Man α1-3) (GlcNAc β1-2 (GlcNAc β1-4) (GlcNAc β1-6) Man α1-6) Man β1-4 GlcNAc β1-4 GlcNAc corresponding to M15 were significantly reduced in the chromatogram of (D); M2 (Man α1-3 (Man α1-6) Man β1-4 GlcNAc β1-4 GlcNAc), M4 (GlcNAc β1-4 (Man α1-3) (Man α1-6) Man β1-4 GlcNAc β1-4 GlcNAc), M5 (GlcNAc β1-2 Man α1-3 (GlcNAc β1-2 Man α1-6) Man β1-4 GlcNAc β1-4 GlcNAc), and M7 (GlcNAc β1-2 (GlcNAc β1-4) Man α1-3 (GlcNAc β1-2 Man α1-6) Man β1-4 GlcNAc β1-4 GlcNAc) were gradually increased; and N-acetylglucosamine at the terminal portion was cleaved off.

In addition, it was confirmed that, in the case of a derivative obtained by treating OM with sialidase (hereinafter, S-OM), two kinds of sialic acid-binding oligosaccharides, M18 (NeuAc α2-3 or 6 Gal β1-4|GlcNAc β1-4 (GlcNAc β1-2 (GlcNAc β1-4) Man α1-3) (GlcNAc β1-2 (GlcNAc β1-4) Man α1-6) Man β1-4 GlcNAc β1-4 GlcNAc) and M20 (NeuAc α2-3 or 6 Gal β1-4|GlcNAc β1-4 (GlcNAc β1-2 (GlcNAc β1-4) (GlcNAc β1-6) Man α1-3) (GlcNAc β1-2 (GlcNAc β1-4) Man α1-6) Man β1-4 GlcNAc β1-4 GlcNAc) were shifted to M16 (Gal β1-4|GlcNAc β1-4 (GlcNAc β1-2 (GlcNAc β1-4) Man α1-3) (GlcNAc β1-2 (GlcNAc β1-4) Man α1-6) Man β1-4 GlcNAc β1-4 GlcNAc) and M17 (Gal β1-4|GlcNAc β1-4 (GlcNAc β1-2 (GlcNAc β1-4) (GlcNAc β1-6) Man α1-3) (GlcNAc β1-2 (GlcNAc β1-4) Man α1-6) Man β1-4 GlcNAc β1-4 GlcNAc) by cleavage of NeuAc at the terminal portion.

### Example 8

### Comparative Test of Antigenicity of Glycan-Modified OM

<8-1> Mouse Immunization and Antiserum Collection

OM and its exoglycosidase derivatives (G-OM, M-OM, N-OM and S-OM) were used as antigens for antibody production, while 6-wk aged BALB/c mice were used for the production of antibodies to the respective antigens. Each antigen (10 μg/mouse) was intraperitoneally injected to five mice for each test group at an interval of two weeks a total of two times. The mice were boost-immunized with OM (10 μg) as an antigen one week after the final immunization, and the antiserum collection was conducted five days after immunization. The antiserum was stored at −20° C. before the measurement of antibody titer.

<8-2> Measurement of Total IgE Production

The total IgE content against each antigen existing in the serum was measured using a sandwich ELISA kit (BD Biosciences, Franklin Lakes, N.J., USA) for mouse IgE measurement according to the manufacturer's instruction. That is, the coating antibody (anti-IgE) was dispensed into each well of flat-bottomed microtiter plate (Nunc., USA) at 100 μL per well using bicarbonate buffer (pH 9.4), and allowed to adhere to the well at 4° C. for 16 hours. Each well was washed three times with PBS-Tween 20 (0.05%; PBST), blocked using 3% skim milk, and then again washed with PBST. Each of the prepared serums was diluted to 50-fold, and added to each well, followed by reaction at room temperature for 2 hours. After washing, the reaction with the secondary antibody to mouse IgE and the HRP conjugate was conducted. TMB was used as a substrate for HRP action. For colorimetric measurement, the reaction was stopped using 2 N H₂SO₄, and the absorbance was read at 450 nm. The total IgE content in the serum was determined by inserting the measured value a standard curve using an IgE standard material.

As a result of investigating the effect on IgE production by OM and derivatives thereof obtained by the treatment of OM with exoglycosidases, the IgE production by the derivatives were reduced compared with the control group of OM, and especially, N-OM immunization showed a significantly lower IgE production compared with OM immunization, verifying that allergic antigenicity to promote the IgE production was largely lost (see FIG. 12). This result means that the glycan portion of OM plays an important role in the IgE production in association with the allergenic antigenicity of OM, and the allergic antigenicity of OM can be regulated by glycan control.

<8-3> Tests for OM Stimulation and Cytokine Measurement and Stimulation on Lymphocytes

The functions of effector B cells involved in humoral immunity are controlled by cytokines having a Th1 bias, such as IL-2, IFN-γ, and GM-CSF, produced by Th1 cells or by IL-4, IL-5, IL-6, and IL-10, produced by Th2 type helper T cells. These cytokines are involved in the differentiation and proliferation of B cells into effector cells producing several antibodies against respective antigens. That is, the production of IgE antibody to antigens entering the body may be mainly illustrated by cytokines (IL-4, IL-5, IL-6, and IL-10) produced by Th2 cells.

The spleen was taken from the immunized mouse or normal mouse, and the cell density was adjusted to 3×10⁶ cells/well, and dispensed on the 24-well culture plate. OM, G-OM, M-OM, N-OM and S-OM samples with a final antigen concentration of 10 μg/mL were added to each well in which the spleen cells were dispensed, and the cells were cultured at 37° C. in a 5% CO₂ incubator for 72 hours. After the completion of culturing, the amount of IL existing in the culture supernatant was investigated using an ELISA kit (BD Biosciences).

As a result of measuring the production of IL-4 produced after the spleen cells obtained from the mice immunized with OM and its derivatives were re-stimulated with respective antigens, as shown in FIG. 13, the derivative-immunized mice showed a reduced IL-4 production as compared with the control group of OM, and especially, N-OM showed a significantly reduced IL-4 production, resulting in the same tendency in production considering the IgE production. Therefore, it is determined that the derivatives prepared by the modification of glycan from OM, especially, N-OM, is modified to have a structure causing little OM allergy, while inhibiting the type 1 immune hypersensitivity response.

The inventive concept established a method for reducing antigenicity of a glycoprotein of an allergenic food by removing a sugar linked to the glycoprotein, and thus, a low antigenic glycoprotein can be prepared. Therefore, the glycoprotein of the inventive concept has a very low probability of causing allergy, and maintains inherent nutritional values intact, leading to the preparation of high-nutrient foods and cosmetic composition, and thus the inventive concept is highly industrially applicable.

Although certain exemplary embodiments and implementations have been described herein, other embodiments and modifications will be apparent from this description. Accordingly, the inventive concept is not limited to such embodiments, but rather to the broader scope of the presented claims and various obvious modifications and equivalent arrangements.

What is claimed is:

1. A method for preparing a low antigenic food, the method comprising removing a sugar linked to a glycoprotein of an allergenic food.

2. The method of claim 1, wherein the allergenic food is any one selected from the group consisting of whole eggs, beans, and peanuts.

3. The method of claim 1, wherein the glycoprotein is at least one selected from the group consisting of ovalbumin, ovomucoid, ovotransferrin, β-conglycinin, Ara h1, and Ara h2.

4. The method of claim 3, wherein the glycoprotein is ovalbumin or ovomucoid.

5. The method of claim 1, wherein the sugar is at least one selected from the group consisting of mannose, galactose, N-acetylglucosamine, and N-acetylneuraminic acid.

6. The method of claim 1, wherein removing a sugar linked to a glycoprotein is performed by treating the glycoprotein with an exoglycosidase.

7. The method of claim 6, wherein the exoglycosidase is at least one selected from the group consisting of mannosidase, galactosidase, N-acetylglucosaminidase, and sialidase.

8. A low antigenic food prepared by the method of claim 1.

9. The food of claim 8, wherein food is any one selected from the group consisting of whole eggs, beans, and peanuts.

10. A method for preparing a low antigenic glycoprotein, the method comprising removing a glucose linked to a glycoprotein selected from the group consisting of ovalbumin, ovomucoid, ovotransferrin, β-conglycinin, Ara h1, and Ara h2.

11. A low antigenic glycoprotein prepared by the method of claim 10.

12. A low antigenic food composition comprising the low antigenic glycoprotein of claim 11 as an active ingredient.

13. A low antigenic cosmetic composition comprising the low antigenic glycoprotein of claim 11 as an active ingredient.
